(19) United States
(12) Patent Application Publication
(10) Pub. No.: US 2026/0047774 A1
(43) Pub. Date: Feb. 19, 2026

(54) DRINKING BOTTLE WITH INTEGRATED BIOMETRIC SENSORS

(71) Applicant: Phillip Olla, Detroit, MI (US)

(72) Inventor: Phillip Olla, Detroit, MI (US)

(21) Appl. No.: 19/300,777

(22) Filed: Aug. 15, 2025

Related U.S. Application Data
(60) Provisional application No. 63/683,289, filed on Aug. 15, 2024.

(51) Int. Cl. A61B 5/08; A61J 9/00; B65D 1/02; B65D 81/38

(52) U.S. Cl. CPC A61B 5/082; A61J 9/00; B65D 1/02; B65D 81/3841; A61J 2200/70; B65D 2203/10

(57) ABSTRACT

A drinking bottle including a bottle body, a lid or a cap, and an electronic nose sensor. The bottle body has a base, at least one sidewall extending from the base, a neck extending from the at least one sidewall, and an opening at a top of the neck that fluidly connects an internal volume of the bottle body with an exterior environment. The lid or cap is configured to engage with the neck of the bottle body to selectively close off and seal the opening of the bottle body for adding or removing liquid contents within the volume of the bottle body. The electronic nose sensor is in electrical communication with a power source and is configured to collect and analyze a plurality of breath biomarkers. The electronic nose sensor and the power source are both disposed on one of the bottle body or the lid or cap.

### RELATED APPLICATIONS

This application claims priority benefit of U.S. Provisional Application Ser. No. 63/683,289, filed Aug. 15, 2024, the contents of which are hereby incorporated by reference.

### FIELD OF THE INVENTION

The present invention in general relates to a bottle for containing and dispensing liquids for drinking, and more particularly to a drinking bottle equipped with sensor technology to collect health and wellness data based on biometric inputs. Such drinking bottles are also called Smart Bottles.

### BACKGROUND OF THE INVENTION

A healthy lifestyle is a way of living that allows an individual to lower the risk of diseases, stay fit, and increase or maintain immunity towards infections. World Health Organization (WHO) defines health as “a complete state of mental, physical, and social well-being not merely the absence of disease.” A healthy lifestyle includes eating a balanced diet and limiting unhealthy foods, exercising regularly, having routine physician examinations for signs of disease, and drinking water to stay hydrated.

A wide range of tools and equipment exist to assist in the pursuit of a heathy lifestyle ranging from technologically advanced devices to simple objects. For example, fitness monitors are technologically advanced wearable devices that include sensors to capture fitness data, exercise data, or vital sign data of the wearer to provide real time health and wellness feedback and, in some cases, wirelessly communicate the captured data to a computer or mobile phone application for tracking the captured data over a period of time. On the other hand, simple drinking bottles for staying hydrated are an extremely popular health and wellness tool, typically including a bottle body for storing liquid and a closure unit with a movable or completely removable lid, sometimes provided with a spout or straw, for drinking liquid from the drinking bottle.

It has been found that the breath of humans and animals contains a large number of volatile organic components (VOCs). Many of these VOCs are now recognized as biomarkers of disease, so that a breath test for VOCs offers a safe and non-invasive approach to disease detection. The most abundant breath VOCs (e.g., acetone, isoprene, and pentane) are present in nanomolar concentrations (10⁻⁹ mol/L) but the majority of breath VOCs are present in much lower concentrations, e.g., picomolar (10⁻¹² mol/L), or even as low as parts per trillion. Given the low concentration of VOCs in breath, risks of contamination of the breath sample during the collection process, and challenges due to the background matrix of breath (such as moisture and humidity, flow rate, and temperature) use of analysis of VOCs contained in human breath for use in health and wellness monitoring is not widespread.

With the advent of health and technology integration, there is a growing demand for devices that not only serve basic needs but also enhance user health through widespread monitoring and data analysis. Existing solutions require multiple devices or lack features and capabilities to provide a detailed analysis of an individual's health and wellness indicators, limiting user convenience and functionality

Thus, there is a need for a drinking bottle for containing and dispensing a hydrating liquid that is equipped with sensor technology to collect a wide array of health and wellness data based on biometric inputs.

### SUMMARY OF THE INVENTION

The present invention provides a drinking bottle that includes a bottle body, a lid or a cap, and an electronic nose sensor. The bottle body has a base, at least one sidewall extending from the base, a neck extending from the at least one sidewall, and an opening at a top of the neck that fluidly connects an internal volume of the bottle body with an exterior environment. The lid or cap is configured to engage with the neck of the bottle body in order to selectively close off and seal the opening of the bottle body for adding or removing liquid contents within the volume of the bottle body. The electronic nose sensor is in electrical communication with a power source and is configured to collect and analyze a plurality of breath biomarkers. The electronic nose sensor and the power source are both disposed on one of the bottle body or the lid or cap.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings wherein:

FIG. 1 shows a partial cross-section view of a drinking bottle according to embodiments of the present invention; and

FIG. 2 a partial cross-section view of a drinking bottle according to embodiments of the present invention.

### DESCRIPTION OF THE INVENTION

The present invention has utility as a drinking bottle for containing and dispensing a hydrating liquid that is equipped with sensor technology to collect a wide array of health and wellness data based on biometric inputs.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

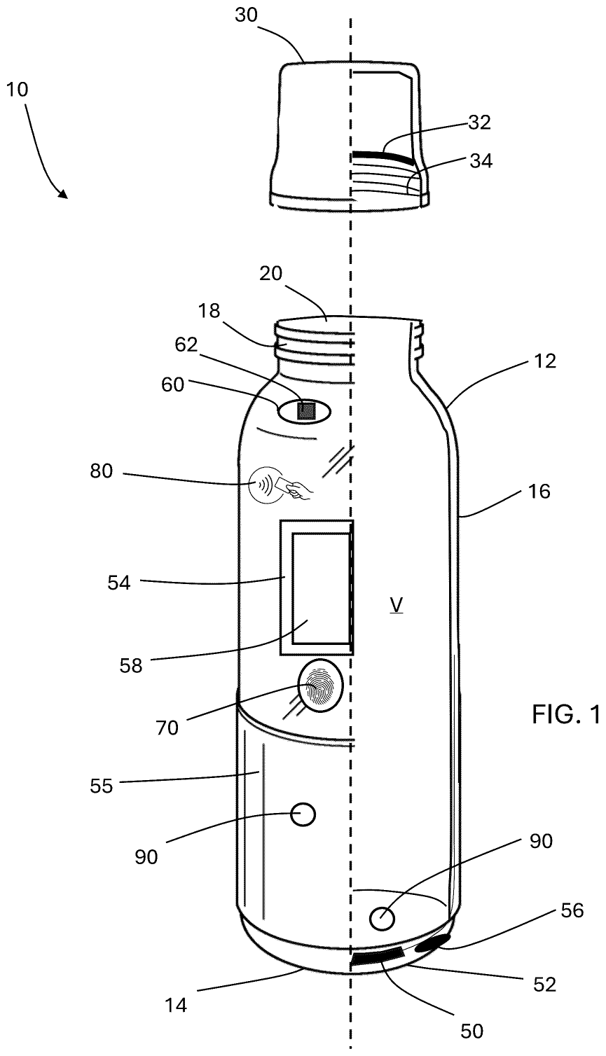

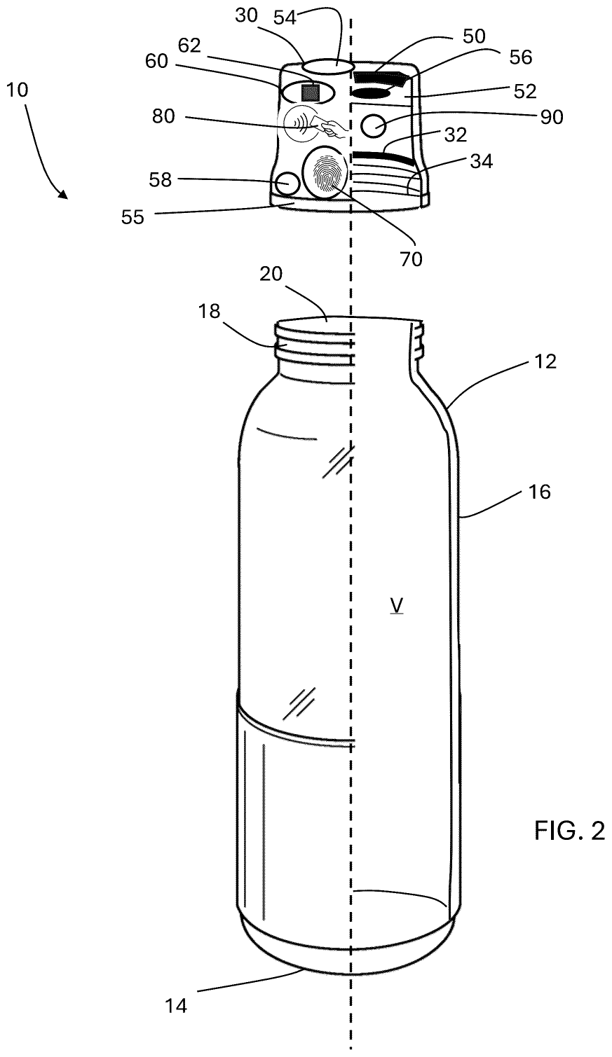

FIGS. 1 and 2 show a drinking bottle 10 according to embodiments of the invention, which is intended for refilling and for repeated use. The drinking bottle 10 has a bottle body 12, which is designed as a double-shell bottle body for thermal insulation. The bottle body 12 has a base 14, at least one sidewall 16, a neck 18, and an opening 20 therein that allows the internal volume V of the bottle body 12 to fluidly communicate with the exterior environment. According to some embodiments, the neck 18 is threaded. According to embodiments, the bottle body 12 is formed of a food safe material such as a metal like stainless steel, or aluminum; glass; or BPA-free plastic. According to embodiments, the bottle body 12 is formed of a thermally insulated double shell. According to embodiments, the bottle body 12 is vacuum insulated, meaning that it includes a space within two walls of metal or other material that is a partial void of air, allowing limited heat transfer to take place between the inside of the vessel and the wall between it and the outside.

The drinking bottle 10 also includes a lid or a cap 30 that is configured to engage with the neck 18 of the bottle body 12 in order to selectively close off and seal the opening 20 of the bottle body 12 for adding or removing liquid contents within the volume V of the bottle body 12. According to some embodiments, the cap 30 has a plurality of threads 34 that are configured to threadably engage with the threaded neck 18. According to embodiments, the threads on the threaded neck 18 are positioned on an exterior surface of the neck 18, in which case the threads on the cap 30 are positioned on an interior surface of the cap 30. According to other embodiments, the threads on the threaded neck 18 are positioned on an interior surface of the neck 18, in which case the threads on the cap 30 are positioned on an exterior surface of the cap 30. According to embodiments, the cap 30 is formed of a food safe material such as metal, plastic or a combination thereof. According to embodiments, the cap 30 includes a gasket 32 that is configured to seal between the neck 18 of the bottle body 12 and the cap 30.

According to embodiments, drinking bottle 10 is a smart bottle in that it includes electronics and sensor technology to collect a wide array of health and wellness data based on biometric inputs. According to embodiments, the electronic elements of the drinking bottle 10 are arranged in the cap 30 or in the bottle body 12. According to embodiments, the electronic elements of the smart drinking bottle 10 are configured to collect biomarker information such as breath biomarkers, fingerprints, and vital signs. According to embodiments, the electronics of the drinking bottle 10 include a power source 50 and an electronic nose sensor 60 in electrical communication with a power source 50. The sensors and various electrical components are connected to the power source 50 via electrical wires that pass within the wall of the bottle body 12.

The electronic nose sensor 60 is configured to collect and analyze a plurality of breath biomarkers. An electronic nose (or E-Nose) is an aroma analyzer that uses mechanical and electronic components to emulate the human olfactory system. Unlike conventional aroma analysis methods under the lab environment, E-Nose is developed for applications demanding quick measurement while avoiding the subjectivity of humans. According to embodiments, the electronic nose sensor 60 is positioned on an exterior surface of the bottle body 12 or the cap 30, so that the sensor is readily accessible by a user of the bottle 10. According to embodiments the electronic nose sensor 60 is a port that is configured to receive a breath sample of a user for the sensor 60 to analyze for the breath biomarkers. According to embodiments, the plurality of breath biomarkers are indicative of various health metrics including ketosis, halitosis, diabetes, and athletic performance. According to embodiments, the plurality of breath biomarkers are volatile organic compounds (VOCs) from a user's breath. The electronic nose sensor 60 is in electronic communication with a power source 50. In some inventive embodiments, a detachable microfluidics chamber 62 is provided in respiratory communication with the breath of a user and the electronic nose sensor 60. The microfluidics chamber 62 is readily removed and shipped to a laboratory for additional testing to confirm a finding provided by smart drinking bottle 10 or afford qualitative or quantitative analysis for analytes beyond that provided by smart drinking bottle 10.

According to embodiments, the power source 50 is a rechargeable battery. According to embodiments, the power source 50 is positioned in a sealed compartment 52 provided in one of the cap 30 or the bottle body 12. The sealed compartment 52 is separated from the contents containing interior volume V of the bottle body 12 or the area of the cap 30 that could come in contact with a liquid. This separation protects the components contained within the sealed compartment 52 against moisture, but also achieves thermal insulation. According to embodiments, the rechargeable battery 50 is configured to be charged by a solar cell 54 positioned on an exterior surface of the drinking bottle 10. According to embodiments, the rechargeable battery 50 is configured to be charged by an energy from human skin potentials when in contact with an exterior surface of the drinking bottle 10. According to such embodiments, the drinking bottle 10 includes at least one thermoelectric generator device 55 positioned on an exterior surface of the bottle 10 that converts heat emitted from human skin to electrical power to recharge the power source 50.

According to embodiments, the sealed compartment 52 also contains an electronic evaluation and communication unit 56 that serves the purpose of recording any information collected from the sensors of the drinking bottle 10 in order to carry out an analysis on this collected information. Examples of the collected information include biomarker information from breath, fingerprint information, vital sign information, and condition data. This analysis can be carried out by electronic components of the drinking bottle itself. Preferably, however, the drinking bottle only has a simple integrated circuit with low computing power and low energy consumption and communicates via a wireless interface such as Bluetooth Low Energy with an external device such as a mobile phone on which a more intensive analysis process can take place.

According to embodiments, the drinking bottle 10 additionally includes indicator 58 in electrical communication with the power source 50. According to embodiments, the indicator 58 is an LED (as in FIG. 2), a display screen (as in FIG. 1), or a combination thereof. According to embodiments, the indicator 58 is positioned on an exterior surface of the bottle body 12 or the cap 30, so that the indicator is readily viewable by a user of the bottle 10.

According to embodiments, the drinking bottle 10 additionally includes a fingerprint-based biometric sensor 70 in electrical communication with the power source 50. According to embodiments, the fingerprint-based biometric sensor 70 is positioned on an exterior surface of the bottle body 12 or the cap 30, so that the sensor is readily accessible by a user of the bottle 10. According to embodiments, the fingerprint based biometric sensor 70 is configured to receive and authenticate a user's fingerprint prior to allowing access to information displayed by the indicator 58, use of the electronic nose sensor 60, or other features of the drinking bottle 10. According to embodiments, the fingerprint based biometric sensor 70 is also configured to collect biometric vital information indicative of various health metrics, such as heart rate, body temperature, etc.

According to embodiments, the drinking bottle 10 additionally includes a tap payment enabled sensor 80. According to embodiments, the tap payment enabled sensor 80 is uses Near Field Communication (NFC) and Radio-frequency identification (RFID) technology, a short-range wireless communication system that allows data exchange between devices in close proximity. Once a user has linked their credit card or blockchain account to the tap payment enabled sensor 80 of the drinking bottle 10, the user is able to simply tap the drinking bottle 10 on a contactless payment terminal to pay for goods or services via credit card or blockchain. According to embodiments, the tap payment enabled sensor 80 requires authentication from a fingerprint-based biometric sensor 70 prior to allowing payment using the tap payment enabled sensor 80.

According to some embodiments, the drinking bottle 10 includes at least one additional sensor 90 configured to collect condition data, such as a temperature sensor to measure the temperature of the liquid contained in the bottle body 12, an accelerometer, a GPS sensor, a step counter, etc.

As a person skilled in the art will recognize from the previous detailed description and from the figures and claims, modifications and changes can be made to the preferred embodiments of the invention without departing from the scope of this invention defined in the following claims.

1. A drinking bottle comprising:
a bottle body having a base, at least one sidewall extending from the base, a neck extending from the at least one sidewall, and an opening at a top of the neck that fluidly connects an internal volume of the bottle body with an exterior environment;
a cap configured to engage with the neck of the bottle body in order to selectively close off and seal the opening of the bottle body for adding or removing liquid contents within the volume of the bottle body;
an electronic nose sensor in electrical communication with a power source, the electronic nose sensor configured to collect and analyze a plurality of breath biomarkers;
wherein the electronic nose sensor and the power source are both disposed on one of the bottle body or the cap.

2. The drinking bottle of claim 1 wherein the bottle body and the cap are each independently formed of metal or plastic.

3. The drinking bottle of claim 1 wherein the bottle body is formed of a thermally insulated double shell.

4. The drinking bottle of claim 1 wherein the cap includes a gasket that is configured to seal between the neck of the bottle body and the cap.

5. The drinking bottle of claim 1 wherein the electronic nose sensor is positioned on an exterior surface of the bottle body or the cap.

6. The drinking bottle of claim 1 wherein the plurality of breath biomarkers are indicative of various health metrics including ketosis, halitosis, diabetes, and athletic performance.

7. The drinking bottle of claim 1 wherein the plurality of breath biomarkers are volatile organic compounds (VOCs) from a user's breath.

8. The drinking bottle of claim 1 wherein the power source is positioned in a sealed compartment of the bottle body of the cap.

9. The drinking bottle of claim 1 wherein the power source is a rechargeable battery.

10. The drinking bottle of claim 9 wherein the rechargeable battery is configured to be charged by a solar cell positioned on an exterior surface of the drinking bottle or by an energy from human skin potentials when in contact with an exterior surface of the drinking bottle.

11. The drinking bottle of claim 1 further comprising a fingerprint-based biometric sensor in electrical communication with the power source that is configured to collect biometric vital information indicative of various health metrics.

12. The drinking bottle of claim 1 further comprising tap payment enabled sensor which optionally requires authentication from a fingerprint-based biometric sensor.

13. The drinking bottle of claim 1 further comprising an indicator in electrical communication with the power source.

14. The drinking bottle of claim 13 the indicator is an LED or a display screen.

15. The drinking bottle of claim 1 further comprising a microfluidics chamber.

16. The drinking bottle of claim 1 further comprising a communication unit in communication with the power source, the communication unit configured to communicate collected biomarker information to a mobile application.

17. A method of collecting wellness data from a user comprising:
the user exhaling into the electronic nose sensor of a bottle of claim 1; and
receiving the wellness data.

18. The method of claim 17 further comprising removing a microfluidics chamber and shipping the microfluidics chamber to a laboratory to receive the wellness data.

19. A drinking bottle comprising:
a bottle body having a base, at least one sidewall extending from the base, a neck extending from the at least one sidewall, and an opening at a top of the neck that fluidly connects an internal volume of the bottle body with an exterior environment;
a lid configured to engage with the neck of the bottle body in order to selectively close off and seal the opening of the bottle body for adding or removing liquid contents within the volume of the bottle body;
an electronic nose sensor in electrical communication with a power source, the electronic nose sensor configured to collect and analyze a plurality of saliva biomarkers;
wherein the electronic nose sensor and the power source are both disposed on one of the bottle body or the cap.

20. A nursing bottle, for feeding a baby with an electronic nose sensor in electrical communication with a power source, the electronic nose sensor configured to collect and analyze a plurality of saliva biomarkers, wherein the electronic nose sensor and the power source are both disposed on one of the bottle body or the cap.
